(19) United States
(12) Patent Application Publication
(10) Pub. No.: US 2009/0043267 A1
(43) Pub. Date: Feb. 12, 2009

(54) Portable Self-Contained Bowel Lavage System

(76) Inventor: Jenifer Jackson, Birmingham, AL (US)

Correspondence Address: BUSH INTELLECTUAL PROPERTY LAW GROUP, LLC, P.O. BOX 381146, BIRMINGHAM, AL, 35238, US

(21) Appl. No.: 11/817,367

(22) PCT Filed: Jun. 29, 2006
(86) PCT No.: PCT/US06/25425

§ 371 Date: Aug. 29, 2007

Related U.S. Application Data
(60) Provisional application No. 60/695,979, filed on Jul. 1, 2005.

(51) Int. Cl. A61M 31/00

(52) U.S. Cl. 604/275

(57) ABSTRACT

A portable, self-contained bowel lavage system for supporting a person in a sitting or squatting position during an enema, comprising a housing (21) having a seat sloping to an opening (24) over a toilet (25) and a splash-guard enclosure (23) upon which the buttocks are pressed to direct effluent into the toilet receptacle. The housing (21) is supported by legs (27) and has a seatback (26) to support a user, both of which may be removed for storage or transport. Water is supplied from a water source to a water handling system, most of which is housed within the waterproof housing (21). Water pressure is controlled by a fixed flow-control valve (52). The device has two water passageways, a first communicating with a sprayer (49) for clean-up and a second communicating through an on/off valve (50), a water filter (53), and a back-flow check-valve (55) to a rectal nozzle (56) extending from the splash-guard enclosure (23).

### TECHNICAL FIELD

The present invention relates to a portable, self-contained bowel lavage system, and more particularly, to a toilet supported and drained device providing a comfortable, physiologically correct position for successful lavage of the lower bowel.

### BACKGROUND ART

Enema boards have been available for use for many years, presenting an opportunity for home cleansing of the colon. U.S. Pat. Nos. D264,881, D282,781, and D367,705 disclose devices that are long in length for the user to lay flat upon. These devices require a bathroom of sufficient size to accommodate a chair and a five gallon bucket that must be placed well above the toilet for gravity flow. U.S. Pat. No. 4,628,554 discloses a form of enema board that is long and flat for the user to lay prone upon. The '554 device provides its own support, thus eliminating the need for a chair, but still requires sufficient space and a large bucket. For all these prior art systems, water filtration must be arranged separately, and a place to position a five gallon bucket considerably higher than the device must be contrived inasmuch as a shelf to support a water-filled bucket is not generally available in a bathroom. Substantial strength is necessary to lift a large bucket to the appropriate height, which many people to do not possess. Another disadvantage is the variability of the height at which the bucket is placed, which determines the rate of water flow and may result in a flow rate which is potentially injurious. The quality of the water with respect to its purification is also a variable that is not provided for in the prior art. Finally, significant time must be employed to set up, clean up, and put away all items involved. The entire prior art process is unwieldy and, therefore, most enema board owners seldom use them.

The position in which colon cleansing is traditionally attempted, either privately or with professional colonic irrigation, is laying flat, which is an unnatural position for evacuation of the bowels. Furthermore, a flat position provides little relief from cramping and discomfort, which most often precedes the release of hydrated material from the bowel during a lavage. The natural position for evacuation of the bowel is squatting, which opens the sigmoid and rectum angle to allow waste to be eliminated quickly and effortlessly. The squatting position requires holding the weight of the upper body over the knees for balance, thus it is believed that to sit with the torso close to the knees is the most effective position to accomplish release of matter from the colon.

U.S. Pat. Nos. 5,380,275, 6,665,888, and RE38,690 disclose devices whereupon a user must sit upright on a toilet. While providing for a better position, the disadvantage of this prior art is that, because of the amount of time required to lavage the colon, circulation will be cut off to the legs, thereby creating a danger of falling and incurring injury upon arising. Furthermore, while claims of “self-contained” and “portable” are made in U.S. Pat. No. 5,871,463, the necessity remains for additional accouterment such as a treatment table and water filtration. In travel situations, few bathrooms are of a size to accommodate a treatment table, requiring the table to be set up in another room. This typically requires long tubes in a separate travel container for attaching to the water source in the bathroom and the toilet for draining the waste effluent. This consideration, along with the transport of a treatment table and a water filtration system, effectively negates the self-containment and portability aspect of this prior art with respect to its actual function.

Accordingly, what is needed and is not found in the prior art, is a portable, self-contained bowel lavage system that is toilet supported and drained, and provides a comfortable, physiologically correct position for lavage of the lower bowel.

### SUMMARY OF THE INVENTION

An object of the present invention is to provide a relatively small, lightweight, safe, self-contained bowel lavage system by which to lavage the bowel without difficulty, discomfort, or inconvenience.

Another object of the present invention is to provide a structure by which a user is supported in the most comfortable, physiologically correct and safe position to accomplish effective lavage of the bowel.

Another object of the invention is to provide an all-inclusive, self-contained system by which to accomplish effective lavage of the bowel.

Another object of the invention is to provide a compact design that will be universally workable in any bathroom of any size and configuration.

Additional objects of the invention include providing filtration and purification of source water; precise modulation of water flow; prevention of back-flow of waste effluent into the device; and ease of use with respect to assembly, cleaning of the user and the device, disassembly, and storage and/or transport of the device.

The present invention is a portable, self-contained bowel lavage system for supporting a person in a sitting position during an enema, comprising a housing having a seat sloping to an opening over a toilet and a splash-guard enclosure upon which the buttocks are pressed to direct effluent into the toilet receptacle. The rearward end of the housing is supported by legs and has a seatback to support the back of a user, both of which may be removed for storage or transport. The bowel lavage system includes a water handling system, wherein many of the components of the water handling system are encased within the waterproof housing. Water is supplied from a water source to an inlet in the right posterior underside of the housing. Water pressure is reduced by a fixed flow-control valve. The water passes within a first conduit to a sprayer for clean-up and within a second conduit through an on/off valve, through a fixed flow-reducing orifice, through a water filter, and through a back-flow check-valve to a rectal nozzle extending from the splash-guard enclosure.

Unlike professional colonics and enema boards, the present invention allows the user to be in a physiologically correct position for the most effective and most comfortable elimination of waste. The upright configuration of the invention calls for much less space and can be utilized in the smallest of bathrooms. Unlike prior art portable colonic irrigation devices, no additional structure or furniture is needed for the support of the user while receiving treatment. Unlike enema board and gravity-fed systems, the present invention is simple and fast to set up, clean up, and put away, thus permitting an ease of use that exceeds all prior art. The present invention is an all-inclusive system that preferably hooks up to a diverter valve provided to fit on any standard shower or standard bathroom sink faucets, and provides flow from available house water pressure. The invention may include a flexible plastic water container that may be hung over the shower pipe and connected thereto to receive water, thereby allowing the device to be provided with gravity fed water. The invention also provides for reduction and regulation of the water flow, water filtration and the prevention of back-flow. Clean up is easily accomplished with the attached sprayer. Assembly and disassembly take only minutes. The entire invention, which includes all items required for full functionality, can fit into a medium-sized rolling travel case that provides for ease of storage and travel.

### BRIEF DESCRIPTION OF THE DRAWINGS

FIG. 1 is a right side elevational view of the invention supported on a standard toilet.

FIG. 2 is a right side sectional view of the waterproof housing taken along line 2-2 of FIG. 3.

FIG. 3 is a top plan view of the waterproof housing.

FIG. 4 is a bottom view of the waterproof housing.

FIG. 5 is a topside perspective view of the waterproof housing.

FIG. 6 is an underside perspective view of the waterproof housing.

FIG. 7 is an exploded perspective view of the invention.

FIG. 8 is a schematic of the components of the invention for handling water flow.

### BEST MODES FOR CARRYING OUT THE INVENTION

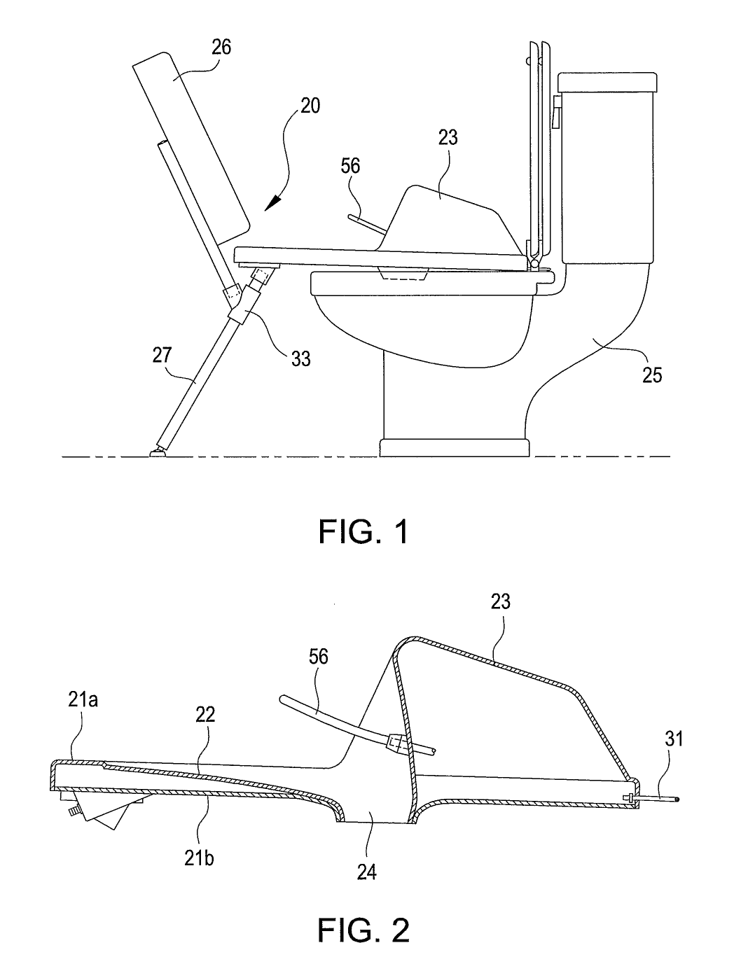

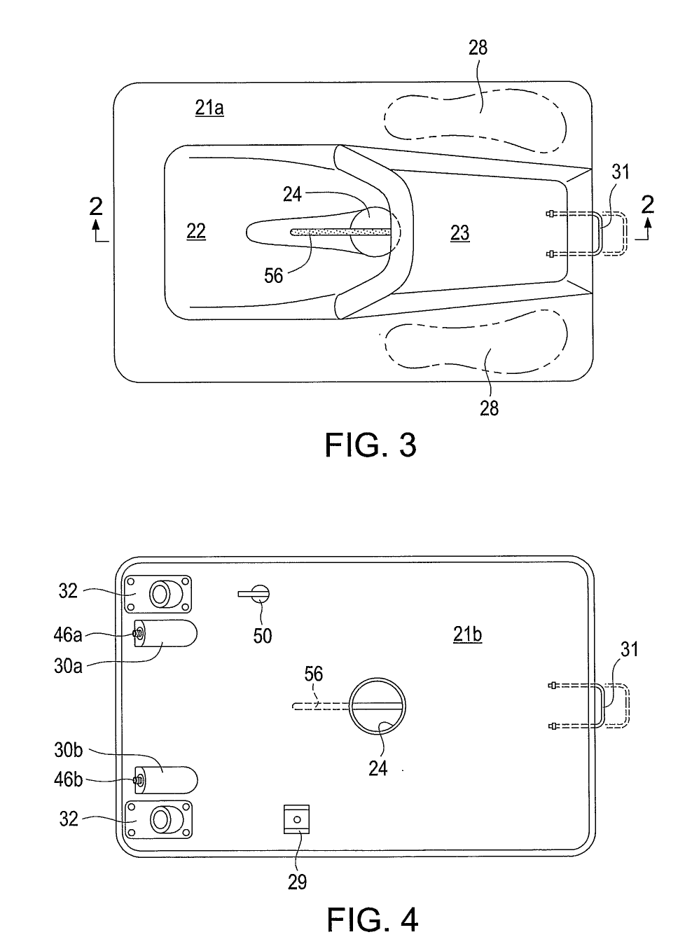

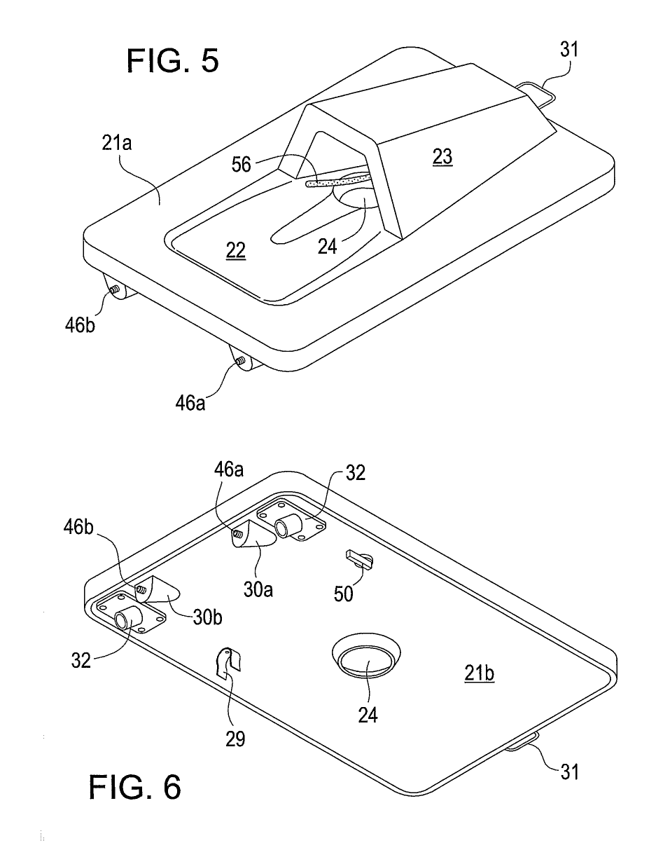

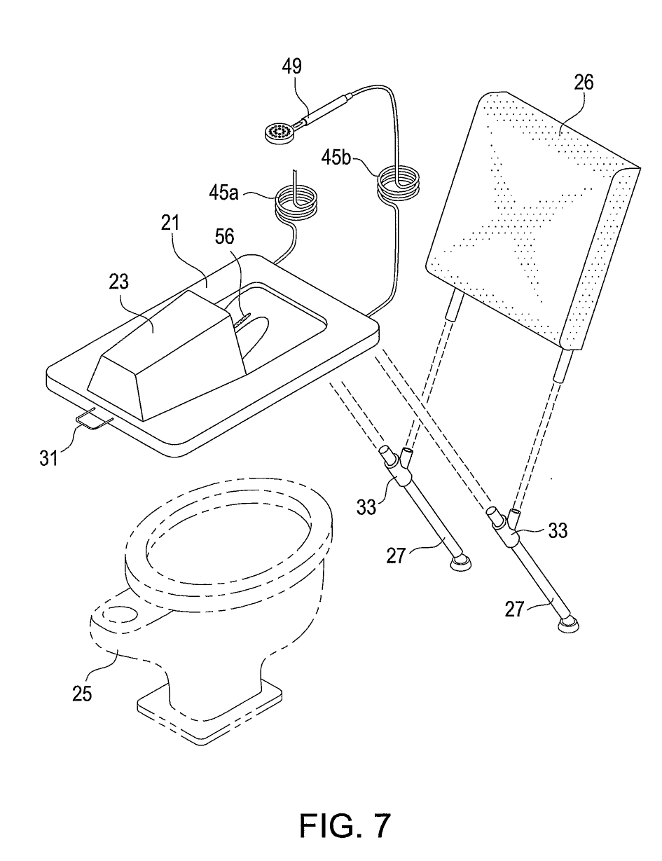

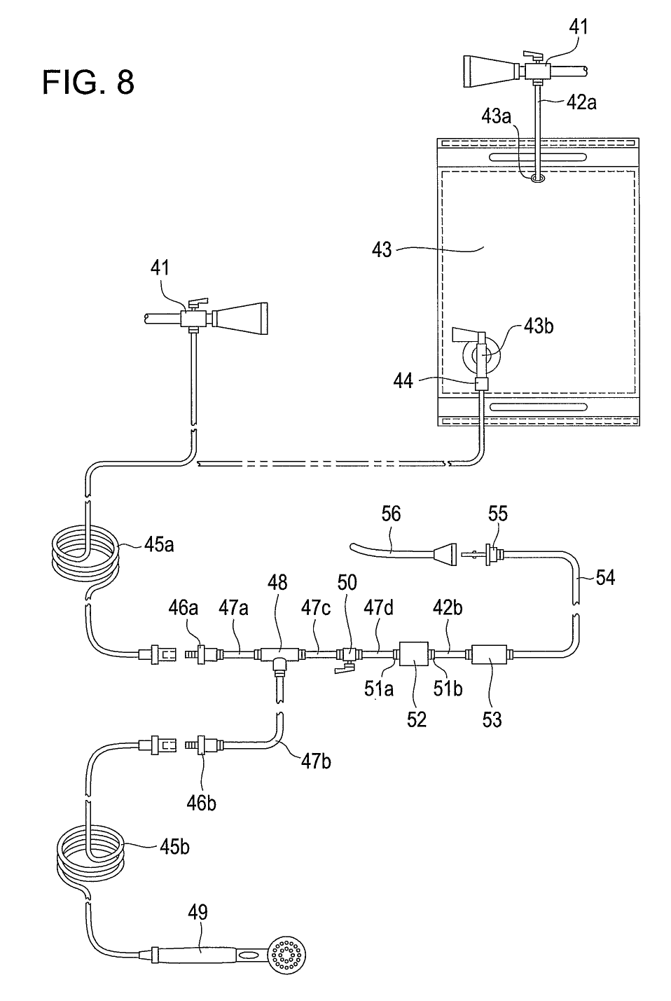

The present application claims priority to U.S. Provisional Patent Application Ser. No. 60/695979, filed Jul. 1, 2005, the disclosure of which is incorporated herein by reference. The preferred embodiment of the invention 20 is shown in attached FIGS. 1-8. The present invention 20 may be disassembled for storage or transport, wherein the disassembled device preferably has a width of about 16 inches, a length of about 22 inches, and a weight of about 30 lbs. or less, and may be stored or transported in a medium-sized, hard-sided rolling travel case (not shown). Once assembled, the invention 20 has a chair-like configuration, shown in FIGS. 1 and 7, which can be placed over a toilet 25. The invention can be supplied with gravity-fed water from a collapsible water container 43 for the purpose of an enema, or from an available water source, such as a sink faucet or shower, for the purpose of a bidet.

The invention 20 preferably comprises a housing 21 having a top surface 21a forming a seat, a bottom surface 21b, a drainage slope 22, a splash-guard enclosure 23, and an opening 24 to be placed over a toilet 25 for drainage and support of the housing 21. The invention 20 preferably further comprises a seatback 26 that may be attached to legs 27 by stainless steel tee fittings 33 affixed to the legs 27, wherein the seatback 26 and rearward end of the housing 21 are supported by the two removable legs 27. The legs 27 may be adjustable in length and preferably support the rearward end of the housing 21 slightly above the rim of the toilet receptacle to improve comfort, drainage, and universal adaptability to varying toilet heights. The top surface 21a includes two foot positions 28 for supporting the feet of a user in a crouched position.

The invention 20 preferably further comprises a retractable stainless steel handle 31, preferably 3.5 inches wide, extending from the front end of the housing 21 to provide a protrusion for fitting between the bolts of the upright toilet seat for lateral stability. In an alternate embodiment, the handle 31 may be hinged. The legs 27 preferably comprise a ⅞ inch outer diameter (OD) and 0.05 inch wall thickness aluminum tubing that may be attached and detached from stainless steel fittings 32 affixed to the bottom surface 21b for support and stability. The seatback 26 may be attached and detached to stainless steel tee fittings 33 affixed to the legs 27, preferably extending the seatback 26 in a 60 degree angle from the legs 27 and, therefore, approximately 30 degrees from the top surface 21a for support, comfort and stability. The invention may further comprise an optional detachable head rest (not shown) for attachment to the seatback 26 for the comfort of the user, and detachable foot rests (not shown) for attachment to the front corners of the housing 21 to provide for proper position and comfort for users of larger stature.

A 10 liter polyvinyl chloride (PVC) collapsible water container 43 may be hung with the lower end approximately 2-3 feet above the height of the housing 21. A 95 durometer 15-foot coiled Polyurethane ¼ inch OD flexible tube 45a may be attached to and detachable from an inlet bulkhead-mount fitting 46a affixed to the right rear underside of the housing 21. A 95 durometer 3 foot coiled Polyurethane ¼ inch OD flexible tube 45b may be attached to and detachable from an outlet bulkhead-mount fitting 46b affixed to the left rear underside of the housing 21 leading to a hand bidet spray wand 49.

The housing 21 is preferably made of plastic or fiberglass and contains a water-tight space in which to house the internal water handling parts of the device. The housing 21 preferably includes a channel 30a on the right rear underside to allow for the mounting of a ¼ inch nickel-plated brass bulkhead push-to-connect fitting 46a into which a short length of hard National Sanitation Foundation (NSF) approved nylon ¼ inch OD tube 47a is inserted, connecting then to an Acetal Copolymer ¼ inch push-to-connect diverter tee 48. Another hard nylon ¼ inch OD tube 47b is inserted into the branch portion of the tee 48 and connects to another ¼ inch nickel-plated brass bulkhead push-to-connect fitting 46b, into the external end of which a 95 durometer 3 foot coiled Polyurethane ¼ inch OD flexible tubing 45b is inserted, connecting to a hand sprayer-bidet attachment 49 to provide a spray for self-cleaning and cleaning the apparatus.

Into the other tube end of the Acetal Copolymer ¼ inch push-to-connect diverter tee 48 is inserted a very short section of hard nylon ¼ inch OD tubing 47c, the other end of which is inserted into a Polypropylene ¼ inch push-to-connect two way straight ball-valve 50 to open, close, or diminish the flow. Another length of hard nylon ¼ inch OD tube 47d is then inserted into the other end of the ball valve 50, leading to an Acetal Copolymer ¼ inch push-to-connect adapter to ¼ inch male National Pipe Taper (NPT) 51a, which is then connected to a ¼ inch female NPT Polypropyl flow-reducing orifice 52, insuring a flow of 1 liter/minute or less. Another Acetal Copolymer ¼ inch push-to-connect adapter to ¼ inch male NPT 51b is then connected to the other ¼ inch female NPT end of the Polypropylene flow-reducing orifice 52, into which is inserted a section of flexible Polyurethane ¼ inch OD tube 42b of sufficient length to be inserted into the inlet ¼ inch push-to-connect end of preferably a Kinetic Degradation Fluxion (KDF) media bacteria-static water filter 53 for the removal of chlorine and impurities from the water. Into the ⅜ inch push-to-connect outlet end of the water filter 53 is inserted a ⅜ inch OD 95 durometer Polyurethane flexible tube 54, which is then connected to the inlet end of a ⅜ inch single barb nylon check valve 55. The outlet end of the check valve 55 is preferably mounted into and protrudes through the wall of the splash-guard enclosure 23 to prevent back flow of waste into the device and has a nipple onto which a flexible rectal nozzle 56 is attached.

The external components of the invention preferably comprise a 10 liter PVC collapsible water container 43 designed to be hung on a shower fixture with the lower end approximately 2-3 feet above the height of the housing 21. Into the top opening 43a of the container 43 is inserted the open end of a 95 durometer clear Polyurethane ¼ inch OD flexible tube 42a attached to a quick disconnect chrome-plated brass water diverter valve 41 to be installed before the shower head. An Acetal Copolymer ⅜ inch straight adapter 44 is attached to the water container outflow 43b to which a 95 durometer 15 foot coiled Polyurethane ¼ inch OD flexible tube 45a is attached which leads to the inlet bulkhead-mount fitting 46a for purposes of an enema. For purposes of bidet cleansing, the 95 durometer 15 foot coiled Polyurethane ¼ inch OD flexible tube 45a will lead directly to a quick disconnect chrome-plated brass water diverter valve 41 (shower or sink) to be quickly attachable and detachable at the water source. This allows for establishing the temperature of the water prior to switching the flow of water to be diverted to the device. The other end of the 95 durometer 15 foot coiled Polyurethane ¼ inch OD flexible tube 45a is attached to and detachable from the inlet bulkhead-mount fitting 46a.

In operation, the user may draw out the retractable handle 31 from the housing 21, and then set it upon the rim of the toilet 25 with the lid and seat raised, and the protruding handle 31 is inserted between the bolts of the raised seat and lid to prevent lateral movement of the device. The user will then insert the legs 27 into the fittings 32 attached to the underside of the housing 21. The seatback 26 is then inserted into the fittings 33 attached to the legs 27.

For the purpose of an enema using the water container 43 as the source of water flow, the user may install the diverter valve 41 to the shower which will remain there for quick connect and disconnect of the tube 42a to the source of water. The user may then hang the 10 liter PVC collapsible water container 43 on the shower fixture with the lower end approximately 2-3 feet above the height of the housing 21. The user may then insert one end of the tube 42a into the top opening 43a of the water container 43, and attach the other end of the tube 42a to the quick disconnect chrome-plated brass water diverter valve 41 at the shower head to fill the container with up to 10 liters of water of an appropriate temperature of approximate body-temperature. An Acetal Copolymer ⅜ inch straight adapter 44 is attached to the water container outflow 43b to which the long coiled tube 45a will be attached to lead to the inlet bulkhead-mount fitting 46a. The user may then open the flow of the valve at the bottom of the water container 43, and then open the flow of the valve 50 located in the bottom 21b of the device to run water through the line that leads to the outlet end of the check valve 55. Once the water has flowed sufficiently through the device to clear the lines of air, the user can turn off the flow at the lower valve 50 and affix the flexible rectal nozzle 56 onto the outflow 55 of the device, straddle the housing 21, and using a lubricant, lower and guide the nozzle 56 into his or her rectum until his or her buttocks are pressed firmly against the rim of the splash guard opening 23. The user may then recline in an upright position upon the padded seatback 26 and draw his or her feet up to one of the two foot positions 28 provided.

The user can then initiate the enema by opening the valve 50 to allow the flow to begin. The gentle flow of purified water will then enter the lower bowel and flow freely in and out as needed, carrying away waste to be drained automatically in the toilet. Nearing the end of the enema, the user can then back off of the nozzle 56 to allow for rinsing of the rectum, and then close off the flow of the water with the valve 50 and initiate a spray of water with the attached hand bidet sprayer 49 to rinse clean both their person and the device. Once the user has finished and arises, he or she can further rinse the underside of the housing 21, then sanitize it with a non-toxic alcohol and water solution, dry it, take it apart, and put it immediately away in a time-span of just a few minutes.

For the purpose of the bidet, the user may either utilize the water diverter 41 or install a quick connect adapter to the faucet of the sink. The user may then affix one end of the coiled tube 45a into the connection of the water source, and the other end of the long coiled tube 45a into the inlet bulkhead-mount fitting 46a. The user then turns on a flow of both hot and cold water to establish a flow of comfortably warm water. The user then diverts the flow to the device and uses the attached sprayer 49 to run the water through that line. The user may then recline in an upright position upon the padded seatback 26, and draw his or her feet up to one of the two foot positions 28 provided to be in the correct position to have a natural elimination. Afterwards, the user may then rinse clean both their person and the device with the attached hand bidet sprayer 49. Once the user has finished and arises, he or she can further rinse the underside of the device, then sanitize it with a non-toxic alcohol and water solution, dry it, take it apart, and put it immediately away in a time-span of just a few minutes.

While the invention has been shown and described in some detail with reference to specific exemplary embodiments, there is no intention that the invention be limited to such detail. On the contrary, the invention is intended to include any alternative or equivalent embodiments that fall within the spirit and scope of the invention as described and claimed herein.

1. An apparatus for lavaging the lower bowel of a user, comprising:
a. a waterproof housing having an upper portion for supporting the buttocks and feet of the user, a lower portion for engaging a toilet receptacle, a rearward end, a splash guard enclosure for engaging the buttocks of the user, and a drainage hole for allowing waste effluent to drain from said housing into the toilet receptacle;
b. a pair of legs mounted to said rearward end of said housing for supporting said rearward end;
c. a seatback mounted to said legs adjacent said rearward end of said housing for supporting the back of the user; and
d. a water handling system for delivering water from a water source to a rectal nozzle, wherein said rectal nozzle is mounted within said splash guard enclosure and extends therefrom for insertion into the user's rectum.

2. An apparatus according to claim 1, wherein said legs are adjustable in length.

3. An apparatus according to claim 1, wherein said legs are detachable.

4. An apparatus according to claim 1, wherein said seatback is detachable.

5. An apparatus according to claim 1, wherein said water handling system comprises an external water handling system and an internal water handling system, wherein said internal water handling system is housed within said housing.

6. An apparatus according to claim 5, wherein said external water handling system comprises:
a. a water container having a support member for supporting said water container above said housing, an inlet for receiving water from a water source, and an outlet for allowing water to flow out of said container; and
b. flexible tubing connecting said water container to a water inflow port in said housing.

7. An apparatus according to claim 6, wherein said external water handling system further comprises:
a. a hand-operated sprayer; and
b. flexible tubing connecting said sprayer to a water outflow port in said housing.

8. An apparatus according to claim 5, wherein said external water handling system comprises:
a. a water diverter for attachment to a source of pressurized water; and
b. flexible tubing connecting said water diverter to a water inflow port in said housing.

9. An apparatus according to claim 8, wherein said external water handling system further comprises:
a. a hand-operated sprayer; and
b. flexible tubing connecting said sprayer to a water outflow port in said housing.

10. An apparatus according to claim 5, wherein said internal water handling system comprises:
a. a water flow splitter in communication with a water inflow port, a water outflow port, and a water outflow valve, wherein said rectal nozzle is mounted to said outflow valve;
b. an on/off valve for controlling water flow between said splitter and said outflow valve;
c. a water flow controller for regulating the rate of water flow to said outflow valve; and
d. a water filter for filtering water flowing to said outflow valve.

11. An apparatus according to claim 1, further comprising a retractable handle in the center anterior of said housing for engagement between the toilet seat bolts for lateral stability.

12. An apparatus for lavaging the lower bowel of a user, comprising:
a. a waterproof housing having an upper portion for supporting the buttocks and feet of the user, a lower portion for engaging a toilet receptacle, a rearward end, a splash guard enclosure for engaging the buttocks of the user, and a drainage hole for allowing waste effluent to drain from said housing into the toilet receptacle;
b. a pair of detachable legs mounted to said rearward end of said housing for supporting said rearward end, wherein said legs are adjustable in length;
c. a detachable seatback mounted to said legs adjacent said rearward end of said housing for supporting the back of the user; and
d. a water handling system for delivering water from a water source to a rectal nozzle, wherein said rectal nozzle is mounted within said splash guard enclosure and extends therefrom for insertion into the user's rectum.

13. An apparatus according to claim 12, wherein said water handling system comprises:
a. a water container having a support member for supporting said water container above said housing, an inlet for receiving water from a water source, and an outlet for allowing water to flow out of said container;
b. flexible tubing connecting said water container to a water inflow port in said housing;
c. a hand-operated sprayer;
d. flexible tubing connecting said sprayer to a water outflow port in said housing;
e. a water flow splitter in communication with said inflow port, said outflow port, and a water outflow valve, wherein said rectal nozzle is mounted to said outflow valve;
f. an on/off valve for controlling water flow between said splitter and said outflow valve;
g. a water flow controller for regulating the rate of water flow to said outflow valve; and
h. a water filter for filtering water flowing to said outflow valve.

14. An apparatus according to claim 12, wherein said water handling system comprises:
a. a water diverter for attachment to a source of pressurized water;
b. flexible tubing connecting said water diverter to a water inflow port in said housing;
c. a hand-operated sprayer;
d. flexible tubing connecting said sprayer to a water outflow port in said housing;
e. a water flow splitter in communication with said inflow port, said outflow port, and a water outflow valve, wherein said rectal nozzle is mounted to said outflow valve;
f. an on/off valve for controlling water flow between said splitter and said outflow valve;
g. a water flow controller for regulating the rate of water flow to said outflow valve; and
h. a water filter for filtering water flowing to said outflow valve.

15. A method for lavaging the lower bowel of a person, comprising the steps of:
a. positioning an apparatus over a toilet receptacle, wherein said apparatus comprises a waterproof housing having an upper portion for supporting the buttocks and feet of the person, a lower portion for engaging the toilet receptacle, a rearward end, a splash guard enclosure for engaging the buttocks of the person, a drainage hole for allowing waste effluent to drain from said housing into the toilet receptacle; a pair of legs mounted to said rearward end of said housing for supporting said rearward end; and a water handling system for delivering water from a water source to a rectal nozzle; wherein said rectal nozzle is mounted within said splash guard enclosure and extends therefrom for insertion into the person's rectum;
b. positioning the person on said upper portion of said apparatus such that the person's buttocks and feet are supported on said upper portion, the person's buttocks are engaged by said splash guard enclosure, the person is leaning forward in a crouched position such that the person's chest is in close proximity to the person's knees; and said rectal nozzle is inserted into the person's rectum; and
c. activating said water handling system to deliver water from the water source to said rectal nozzle, whereby water is expelled from said rectal nozzle into the person's rectum and waste effluent is drained from said housing into the toilet receptacle.
